(19) United States
(12) Patent Application Publication
(10) Pub. No.: US 2026/0232726 A1
(43) Pub. Date: Aug. 13, 2026

(54) USE OF BORON FOR THE TREATMENT OF LEISHMANIASIS DISEASE OF NEURONAL ORIGIN

(71) Applicant: YEDITEPE UNIVERSITESI, Istanbul (TR)

(72) Inventors: Fikrettin SAHIN, Istanbul (TR); Zeynep ISLEK KOKLU, Istanbul (TR); Hazal URAZ, Istanbul (TR); Hatice OZEKEN, Istanbul (TR); Mehmet Hikmet UCISIK, Istanbul (TR)

(73) Assignee: YEDITEPE UNIVERSITESI, Istanbul (TR)

(21) Appl. No.: 19/156,452

(22) PCT Filed: Feb. 20, 2024
(86) PCT No.: PCT/TR2024/050135

§ 371 Date: Aug. 14, 2025

(30) Foreign Application Priority Data
Feb. 20, 2023 (TR) 2023/001878

(51) Int. Cl. A61K 33/22; A61K 45/06

(52) U.S. Cl. CPC A61K 33/22; A61K 45/06

(57) ABSTRACT

A use of boron derivatives as therapeutics in neuroinflammation caused by Leishmania parasites is provided. The objective is to reduce cell viability on astrocytes infected with Leishmania infantum promastigotes and to use sodium perborate tetrahydrate (SPT), a boron derivative, as therapeutics against neuronal Leishmaniasis because it does not show any toxic effect on healthy cells. The experimental results demonstrates that SPT treatment causes a significant activity (reduction) on infection rate of C8-S astrocyte cells infected with L. infantum parasites by reducing the cellular toxicity on astrocytes and that oxidative stress on infected astrocytes is significantly reduced by inducing GSH. Thus, considering the treatment method developed, the success achieved in the use of SPT as therapeutics against neuronal Leishmaniasis lays the groundwork for treatments of other infectious neurodegenerative diseases and enables the design and development of novel preventive and therapeutic methods.

### CROSS REFERENCE TO THE RELATED APPLICATIONS

This application is the national phase entry of International Application No. PCT/TR2024/050135, filed on Feb. 20, 2024, which is based upon and claims priority to Turkish Patent Application No. 2024/000088, filed on Feb. 20, 2023, the entire contents of which are incorporated herein by reference.

### TECHNICAL FIELD

The present invention relates to the use of boron as therapeutics in neuroinflammation caused by Leishmania parasites.

### BACKGROUND

Leishmaniasis is a collective name given for a group of vector-borne diseases caused by protozoan parasites, which are transmitted to humans by the bite of infected female sandflies. Accordingly, the parasite, which exists in amastigote form when the sandfly gets infected by it, develops into a promastigote in the digestive tract of the sandfly and infects the animal or human bitten by the sandfly as promastigote. The promastigote infects macrophages in the body, and it is transformed into amastigotes in macrophages, thereby causing disease.

According to World Health Organization data, Leishmaniasis is widely common in more than 60 countries worldwide, including Turkey and its countries' geography, especially, countries in Southern Europe, the Middle East, and North Africa [1, 2, 3]. Visceral Leishmaniasis (VL), colloquially known as Kala Azar Disease, which is a form of leishmaniasis, can be fatal within two years if not untreated. Leishmaia infantum is the main parasite species responsible for this disease in the geographical region of our country and can cause VL.

As is the case with other parasitic diseases, chemotherapy is the most effective method in the treatment of leishmaniasis. However, the high toxicity values of antiparasitic compounds and the drug resistance acquired by parasites over time limit the applicability of chemotherapy. To improve conventional treatment for such infections, there is an urgent need for more effective and selective drugs or drug formulations with low toxicity. The inadequacy of treatment methods has led scientists to try using novel chemotherapeutics in the field of leishmaniasis.

The brain is an immune-privileged organ which protects the central nervous system through the blood-brain barrier (BBB) from blood-borne pathogens, including parasites [2] However, some intracellular and extracellular parasites, including Leishmania, can cause central nervous system mvas10n to induce inflammation/damage in the brain during infectious disease, but the damage to neural periphery and mechanisms underlying the subsequent effects are currently being elucidated. Studies on central nervous system infection have shown that Leishmania can cross the blood brain barrier, resulting in neurological manifestations, known as “cerebral leishmaniasis”

Boron derivatives are therapeutic agents with antiviral, anticancer and antibacterial, antifungal, and other disease-specific properties [4]. Boric acid, sodium pentaborate pentahydrate, sodium perborate tetrahydrate (SPT) are defined as boron derivatives [5]. Boric acid, disodium octaborate tetrahydrate and sodium pentaborate are included as antimicrobial agents [6]. Boron has a high affinity for ribose, a constituent of several biological molecules of vitality such as ATP, NADH, NADPH, and RNA. Excessive boron impairs protein synthesis, causes mitochondrial dysfunction, and disrupts cell division and development. Boron is also involved in quorum sensing, which is a vital mechanism in microorganisms and is impaired by increased boron concentrations. In medicine, the implementation of boron compounds in the antibiotic industry is considered important in order to overcome increasing resistance to antibiotics [7].

Furthermore, it has been discovered that boron derivatives such as boric acid have anticancer effects. Different boron compounds and structures have the potential to inhibit cancer progression. Boron compounds have shown inhibitory effects on important cellular components such as proteasomes, proteases, and peptidases. Bortezomib, which is a proteasome inhibitor, has shown to suppress both the viability and migration ability of cancer cells. It has been observed that SPT increased early, and late apoptotic markers compared to other boron derivatives. The reason why SPT is more effective than other boron derivatives is that SPT triggers H2O2-mediated apoptosis [5].

Applications based on the mechanism and treatment of neuroinflammation in the brain caused by Leishmania parasites are among innovative applications, and there is no accepted treatment method in the literature. Accordingly, our study for use of boron derivatives for the treatment of neuroinflammation induced by Leishmania is novel and it is included in the literature as pioneer.

Treatment of kala azar (VL—Visceral Leishmaniasis) is largely based on pentavalent antimonials [8]. The increasing resistance acquired by the parasites against antimonials is the biggest problem limiting the success of this chemotherapy. In fact, in the North Bihar region of India, the success of antimony therapy remains below 50% [9, 10].

Pentavalent antimonials are available as sodium stibogluconate (100 mg/ml) and meglumine antimony (85 mg/ml) and can be administered via i.v. (intravenous) or i.m. (intramuscular). In both administration cases, equal potency is observed. Usually, 28-day therapies are applied as 20 mg/kg/day. Duration and dose may vary based on clinical syndrome and parasite type. However, it is recommended that the total daily dose should not exceed 850 mg. Amphotericin B (AmB) is preferred in cases where antimonial resistance is observed. This polyene antibiotic has a cure rate close to 100%; however, it is toxic, shows strong side effects and requires long-term hospitalization of the patient [8].

In recent years, therapy with liposomal formulation has become more preferred, especially in Southern Europe [11]. Miltefosine, unlike other therapies against leishmaniasis, is the first therapy method that provides oral treatment, and a 3-4-week treatment shows a cure at a level of 95-100% [12, 13]. These data (94%) show a similarity to the results of 6-month Amphotericin B therapy (97%). Its biosafety provides an advantage in one use. Despite its potential for treating large numbers of patients, the primary concern is about the compliance of the drug and possible problems of resistance [14]. In summary, pentavalent antimonials (pentostam and glucantime), miltefosine, paromomycin, amphotericin formulated with deoxycholic acid (Fungizone), and amphotericin formulated with liposomes (AmBisome) are currently used in the treatment of leishmaniasis [15]. Except for miltefosine, all other administrations are given via a vein, i.e., intravenous. Furthermore, other treatment limiting challenges are high cost and toxic side effects. Sodium perborate tetrahydrate offers several advantages which will eliminate or reduce these challenges experienced in the treatment of leishmaniasis [16]. Visceral leishmaniasis (VL) is considered to be a systemic disease showing neurological manifestations; however, the involvement of the nervous system during leishmaniasis is underestimated [17]. There is a wealth of clinical information on neurological effects of leishmaniasis in animal models and human case studies, which indicates that both central nervous system (CNS) and ocular manifestations are common and often underreported [18].
WO2021124301 A1, an application known in the state of the art, discloses formulations developed for use in the treatment of ocular disorders such as uveitis.
U.S. Pat. No. 7,078,399 B2, an application known in the state of the art, discloses sulfhydryl rifamycin compositions, production methods of the said compositions and methods of treating diseases by using these compositions.
US2010010082 A1, an application known in the state of the art, discloses ophthalmic solutions and methods of using the said solutions to treat ocular disorders.

### SUMMARY

The objective of the invention includes boron and SPT, a boron derivative, reducing cell viability on astrocytes infected with Leishmania infantum promastigotes, without showing any toxic effect on healthy cells, thereby their use as therapeutics against neuronal Leishmaniasis. Another objective of the invention is to realize an application that can be an alternative to conventional drug therapies, which are applied in the treatment of Leishmaniasis and neurodegenerative diseases and have difficult patient compliance due to side effects.

### BRIEF DESCRIPTION OF THE DRAWINGS

“Use of Boron for the Treatment of Leishmaniasis Disease of Neuronal Origin”, which is realized to fulfill the objective of the present invention, is illustrated in the accompanying figures, in which:

FIG. 1 is a graphical illustration of the effect of SPT on the viability of astrocytes in 24, 48 and 72 hours of treatment at different doses (50-200 μM).

FIG. 2 is a graphical illustration of the effect of SPT on the viability of astrocytes infected with Leishmania infantum promastigotes in 24, 48 and 72 hours of treatment at different doses (50-400 μM).

FIG. 3 is the illustration of Determination of Oxidative Stress by Measurement of Glutathione (GSH) Level in astrocytes infected with Leishmania infantum promastigotes in 24 hours of treatment at different doses of SPT (50-200 μM).

### DETAILED DESCRIPTION OF THE EMBODIMENTS

The subject matter of the invention relates to boron derivatives (preferably sodium perborate tetrahydrate (SPT)) used for the treatment of cerebral Leishmaniasis disease of neuronal origin, thanks to their lethal effect on astrocytes infected with Leishmania parasites, without showing toxicity in healthy cells. Within the scope of the invention, boron derivatives are used for the treatment of cerebral Leishmaniasis disease of neuronal origin, thanks to their lethal effect on astrocytes infected with a parasite, wherein the said parasite is selected from a group consisting of Leishmania spp. (L. Arabica, L. archibaldi, L. aristedesi, L. braziliensis, L. chagasi, L. colombiensis, L. Deanei, L. donovani, L. enrietii, L. equatorensis, L. forattinii, L. garnhami, L. gerbil, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. tarentolae, L. tropica, L. turanica, L. venezuelensis).

Within the scope of the invention, the said boron derivative used for the treatment of cerebral Leishmaniasis disease of neuronal origin is included in a pharmaceutical composition. The said pharmaceutical composition includes extracellular vesicles obtained from astrocytes infected with Leishmania parasites and at least one nano-carrier system (selected from a group including emulsion systems, biological and chemical nanoparticles (polymeric nanoparticles, solid lipid nanoparticles), inorganic nanoparticles (metallic nanoparticles), lipid vesicular systems (liposomes, niosomes and ethosomes), dendrimers, polymer-drug conjugates micelles, and carbon nanotubes). This pharmaceutical composition includes at least one active compound selected from a group including active compounds showing antiparasitic and/or antineoplastic effect, and binary and ternary combinations thereof, as an active substance. It includes at least one agent selected from a group including nitazoxanide, melarsoprol, eflomithine, metronidazol, tinidazole, miltefosine, mebendazole, pyrantel pamoate, thiabendazole, diethylcarbamazine, ivermectin, niclosamide, praziquantel, albendazole, rifampin, amphotericin B, fumagillin, furazolidone, nifursemizone, nitazoxanide, omidazole, paromomycm sulfate, pentamidine, pirimethamine, tinidazole, albendazole, mebendazole, thiabendazole, fenbendazole, triclabendazole, flubendazole, abamectin, diethylcarbamazine, ivermectin, suramin, pyrantel pamoate, levamisole, niclosamide, nitazoxanide, oxyclozanide, monepantel, derquantel, amphotericin B, urea stibamine, sodium stibogluconate, meglumine antimoniate, paromomycin, miltefosine, fluconazole, pentamidine, bisnaphthalimidopropyl (BNIP) derivatives, and binary or ternary combinations and encapsulations thereof, as active compounds showing antiparasitic effect.

Furthermore, pharmaceutical composition includes at least one agent selected from a group including cyclophosphamide, ifosfamide, temozolomide, capecitabine, 5-fluorouracil, methotrexate, gemcitabine, pemetrexed, mitomycin, bleomycin, epirubicin, doxorubicin, etoposide, paclitaxel, irinotecan, docetaxel, vincristine, carboplatin, cisplatin, oxaliplatin, bevacizumab, cetuximab, gefitinib, imatinib, trastuzumab, denosumab, rituximab, sunitinib, zoledronate, abiraterone, anastrozole, bicalutamide, exemestane, goserelin, medroxyprogesterone, octreotide, tamoxifen, bendamustine, carmustine, chlorambucil, lomustine, melphalan, procarbazine, streptozocin, fludarabine, raltitrexed, actinomycin D, dactinomycin, doxorubicin, mitoxantrone, eribulin, topotecan, vinblastine, vinorelbine, afatinib, aflibercept, crizotinib, dabrafenib, interferon, ipilimumab, lapatinib, nivolumab, panitumumab, pembrolizumab, pertuzumab, sorafenib, trastuzumab emtansine, temsorilimus, vemurafenib, ibandronic acid, pamidronate, bexarotene, buserelin, cyproterone, degarelix, folinic acid, fulvestrant, lanreotide, lenalidomide, letrozole, leuprorelin, megestrol, mesna, thalidomide, vincristine, and binary or ternary combinations and encapsulations thereof, as active compounds showing antineoplastic effect in combination with extracellular vesicles and/or nano-carrier systems.

Pharmaceutical composition of the present invention can be administered by at least one administration method selected from a group consisting of parenteral, intravenous, intradermal, subcutaneous, intraperitoneal, topical, intrathecal, intranasal, intracerebroventricular, ocular, vaginal, urethral, transdermal, sublingual, subarachnoid, rectal, periodontal, perineural, peridural, periarticular, oral, intratympanic, intratumor, intrapulmonary, intrasynovial, intramuscular, intraovarian, intrameningeal, intracorporus cavernosum, intracoronary, intracerebral, epidural, cutaneous, buccal, dental administration methods.

A pharmaceutical composition for use in the treatment of Leishmaniasis according to the claims, including an adjuvant which is at least one selected from the group consisting of monophosphoryl lipid A (MPL), cholesterol, CG oligonucleotide-containing aluminum hydroxide, aluminum phosphate, tocopherol, emulsion systems, or binary or more combinations thereof. The said components used as adjuvant are used alone or in combination with the other agents listed above in the treatment of Leishmaniasis.

Inactivated vaccines produced from dead microorganisms do not show antigenic proliferation; for the dead vaccines showing a lower immunogenic response to be able to create immunization, they should be repeated with multiple doses at regular intervals and administered in conjunction with the adjuvant. Adjuvants are substances that are themselves non-immunogenic, and do not form antibodies, but increase and strengthen the immunogenicity of the antigen to which they are administered. In relatively less purified vaccines in which entire dead microorganism is used, some components of the microorganism (such as endotoxins) may act as adjuvants (intrinsic adjuvants). These vaccines, which also contain “intrinsic adjuvants” in addition to the normal adjuvant added to the vaccine, have the effect of increasing the immunity of both their own antigens and other antigens that they are administered together (such as diphtheria and tetanus vaccines containing whole-cell pertussis vaccine) [19].

Monophosphoryl lipid A (MPL®) is the first vaccine adjuvant to achieve clinical and market success since the introduction of aluminum salts in the early 20th century. First, a component of adjuvant system 4 [AS04 (1)], aluminum hydroxide semi-crystalline gels that are hydrostatically adsorbed with MPL, was approved for use in the HBV vaccine Fendrix (2) in patients with renal failure and then for more extensive use (e.g., HPV vaccine Cervarix). Completely aluminum-free formulations, such as adjuvant system 1 containing MPL and QS-21 in liposomal complexes, have achieved similar success as the adjuvant component of Shingrix, which is a varicella zoster vaccine. Since MPL® is a highly purified derivative of the lipopolysaccharide (LPS) component of the cell wall of Salmonella enterica, its success as an adjuvant is recognized mainly in terms of its activity as a TLR4 agonist which directly activates dendritic cells [20].

In biocompatible and biodegradable, spherical (round) polymeric systems with nanometer-micrometer sizes, poly (DL-lactide-co-glycolide) microspheres can adsorb and carry many different types of long antigens. Polylactide co-glycolide (PLG) microparticle is one of the most commonly used polymeric microspheres. By means of the cationic or anionic PLG preparations, various types of antigens (plasmid DNA, recombinant protein, immunostimulant oligonucleotides) are adsorbed and presented to antigen-presenting cells. In this way, a much stronger immune response is obtained compared to aluminum. It has been found that many adjuvants and immunostimulants, such as non-methylated bacterial/viral CpG DNA and oligonucleotides (TLR9), LPSs and derivatives thereof (TLR4), lipopeptides and tripalithoyl-S-glyceryl cysteine (TLR2), imidazoquinolone (TLR7/TLR8) are Toll-like receptor (TLR) agonists [19].

Leishmaniasis is a neglected tropical disease, caused by the protozoan Leishmania (L.) parasites, and is transmitted by the bite of phlebotomine sandflies. It is endemic in over 98 countries per year, including Turkey and six continental regions. There are several studies on central nervous system infection to show that Leishmania can cross the blood-brain barrier, resulting in neurological manifestations, known as “cerebral leishmaniasis”.

In clinical forms of leishmaniasis, its treatment is highly challenging smce conventional antileishmanial therapies have high side effects and drug resistance, low patient compliance, and low absorption and bioavailability due to their lipophilic properties. Therefore, SPT provides advantages for use because SPT has hydrophilic nature, does not show side effects on health cells, and shows high efficacy on parasites.

According to experimental studies, SPT concentrations of 75-200 μM showed a significant reduction on macrophs infected with L. infantum parasites, without showing any toxicity on astrocytes. Also, a significant reduction in parasite numbers was observed after SPT treatment through immune response activated with astrocyte. A decrease in ROS and oxidative stress levels was determined in infected astrocyte cells. As a result, the present invention is the first study to highlight the communication regarding parasite burden between Leishmania parasites and infected astrocytes, and the inflammatory response to cell-specific infections. Neuro-antileishmanial activity in conjunction with neuroinflammatory-based SPT treatment has been shown on astrocytes infected with Leishmania parasites. The promising activity of SPT on the infection model emphasizes the potential of boron in antileishmanial treatment of neuro-inflammatory leishmanial of cerebral origin in future clinical trials.

The present invention relates to use of boron for the treatment of Leishmaniasis disease of neuronal origin (cerebral Leishmaniasis), thanks to its lethal effect on astrocytes infected with Leishmania parasites, without showing toxicity in healthy cells. Within the scope of neuroinflammation-based antileishmanial therapy, Sodium Perborate Tetrahydrate (SPT) is administered to healthy astrocytes and astrocytes infected with parasites. It has been observed that SPT is lethal on astrocyte cells infected with Leishmania parasites but shows almost no side effects on healthy astrocyte cells. Therefore, it has the potential to be an effective drug for Leishmaniasis disease of neuronal origin. The use of SPT and other boron derivatives in astrocytes infected with Leishmania parasites, which have never been used before for the treatment of neuroinflammation induced by Leishmania, features novelty.

Within the scope of experimental studies conducted while developing the invention, it has been shown that SPT treatment at different doses (50-400 μM) for 24, 48 and 72 hours statistically decreased cell viability on astrocytes infected with Leishmania infantum promastigotes and showed no toxic effect on healthy cells. It is graphically presented that the determined effective doses of SPT (50-400 μM) showed no side effects on healthy astrocytes.

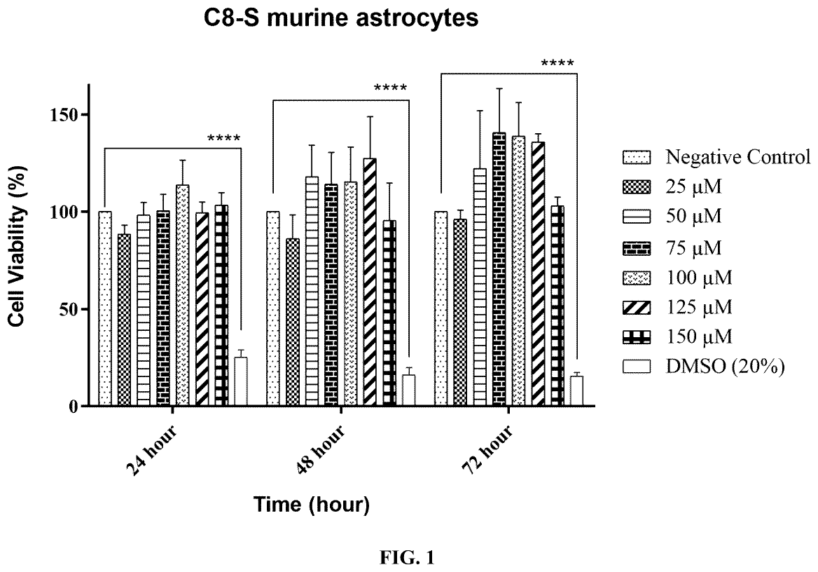

Accordingly, as shown in FIG. 1, when healthy C8-S astrocyte cells were incubated with SPT at concentrations of 25 uM, cell viability of uninfected healthy C8-S astrocytes remained above approximately 90% for 24, 48 and 72 hours. Even when the concentration was increased to 200 μM, cell viability was observed to be above 100% for 24, 48 and 72 hours; astrocyte cell viability was determined to be 100% in cells incubated with 100 μM SPT after 72 hours compared to the negative control group.

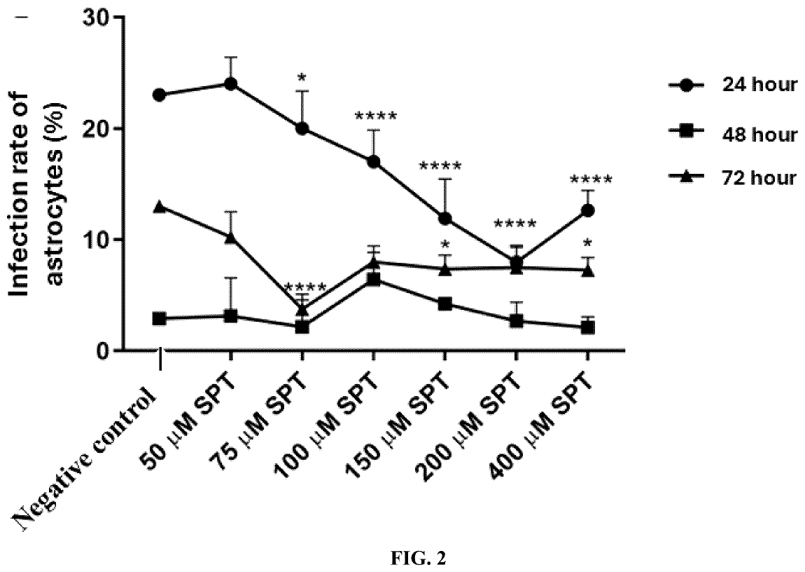

In FIG. 2, after SPT treatment of C8-S astrocyte cells infected with L. infantum parasites for 24, 48 and 72 hours at different doses (50-400 μM), the infection rates of the infected astrocytes are given in percentage. Accordingly, the infection rate of the astrocytes infected with L. infantum parasites was found to be approximately 23%; when incubated with 75, 100, 150, 200, and 400 μM SPT for 24 hours, this rate was found to be 20±3.33% (p 0.05), 17±2.86% (p 0.0001), 11.89±3.56% (p 0.0001), 7.97±1.51% (p 0.0001), and 12.63±1.78% (p 0.0001), respectively, indicating that the infection rate in the cell decreased. After 72 hours of incubation, incubation of astrocytes infected with parasite with 75 μM SPT resulted in a significant decrease up to approximately 4% (P 0.0001) compared to the infection rate of the negative control group (13%). In groups incubated with 100, 150, 200, and 400 μM SPT, the infection rate was close to 7% at 72 hours of incubation.

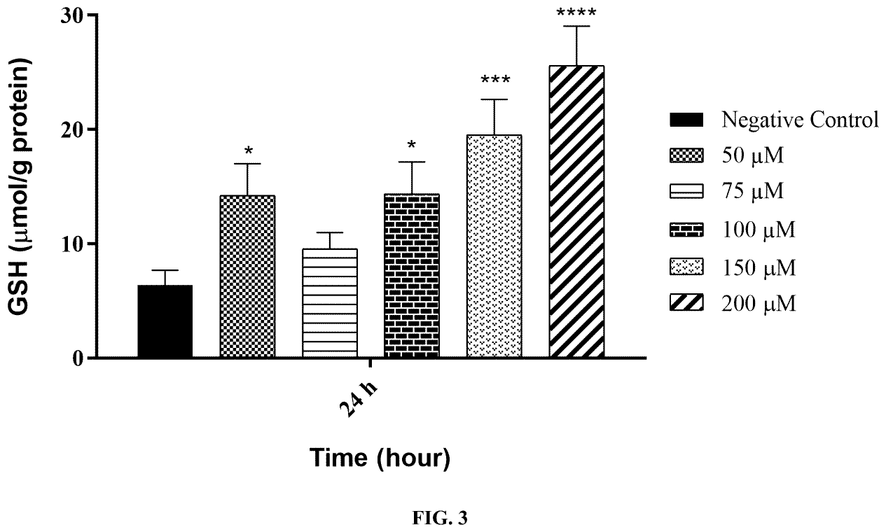

As shown in FIG. 3, the antioxidant capacity of SPT was investigated by measuring GSH levels in astrocytes infected with Leishmania parasites. According to the results obtained, 50, 100, 150 and 200 μM SPT provided a considerable reduction in oxidative stress by significantly inducing GSH levels compared to the negative control (FIG. 3). Therefore, oxidative stress caused by Leishmania infection was significantly reduced by induction of high GSH level by SPT treatment on infected astrocytes.

The results obtained demonstrates that SPT treatment causes a significant activity (reduction) on infection rate of C8-S astrocyte cells infected with L. infantum parasites by reducing the cellular toxicity on astrocytes and that oxidative stress on infected astrocytes is significantly reduced by inducing GSH.

Considering the treatment method developed, the success achieved in the use of SPT and other boron derivatives as therapeutics against neuronal Leishmaniasis will provide a basis for treatments of other infectious neurodegenerative diseases and enable the design and development of novel preventive and therapeutic methods. It is believed that it will provide basis for the molecules that can be an alternative to conventional drug therapies, which are applied in the treatment of Leishmaniasis and neurodegenerative diseases and have difficult patient compliance due to side effects.

### Description of Experimental Study

### 1. Culturing the Parasites

Leishmania infimtum (MHOM/MA/67/TTMA-P263) promastigotes are incubated in RPMI medium (heat inactivated 1 0/4) fetal bovine serum, 2 mM L-glutamine, 20 mM HEPES, 100 U/ml penicillin, 100 μg/ml streptomycin) at 27° C. Parasites reaching the logarithmic phase (10⁶/ml) are made infective.

### 2. Culturing the Astrocyte

Astrocyte C8-S murine cell line (ATCC) is grown as monolayer in RPMI 1640 nutrient medium (2 mM L-glutamine, 100 U/ml penicillin, 100 μg/ml streptomycin) with 10% FBS, heat inactivated in a humidified atmosphere environment with 5% C02 at 37° C. Cells are passaged at 3-day intervals.

3. Treating Astrocytes with SPT and Determining Cell Viability

Cells were seeded in 96-well culture dishes (Coming Glasswork, Coming, NY) in Dulbecco's modified Eagle's medium (DMEM) containing 10% fetal bovine serum (lnvitrogen) and 1% PSA (Biological Industries, Beit Haemek, Israel) at 8.000 cells/well and then treated with SPT, and cell viability levels were measured on days 1, 2 and 3. Cell viability is measured by using 3-(4,5-di-methyl-thiazol-2-yl)-5-(3-carboxy-methoxy-phenyl)-2-(4-sulfo-phenyl)-2H-tetrazolium (MTS)-method (CellTiter96 AqueousOne Solution; Promega, Southampton, UK). 10 μl MTS solution is added onto the cells in 100 μl growth medium and they are incubated in the dark for 2 hours. After the incubation period, viability analysis is obtained by performing absorbance measurement with an ELISA plate reader (Biotek, Winooski, VT) at 490 nm wavelength.

4. Staining the Parasites and Astrocytes with PKH Lipid Membrane Dyes

C8-S astrocytes and L. infantum promastigotes were stained with PKH26 (Red Fluorescent Cell Linker Kit, Sigma-Aldrich) and PKH67 (Green Fluorescent Cell Linker Kit, Sigma-Aldrich), respectively, according to the procedure described previously in the article by Islek et al. (2021). A total cell concentration of 10⁷ cells/ml was used. Before infection, the staining rate of parasites and cells was determined by flow cytometry.

5. Treating the Infected Astrocytes with SPT

The effect of SPT on the proliferation of astrocyte cells infected with parasites is analyzed. In summary, astrocytes are infected with parasites at 37° cat a ratio of 10:1 (parasite: astrocyte). After 3 and a half hours, infected astrocytes are washed with medium to remove the remaining parasites. Infected astrocytes are left to incubate with SPT at different concentration ranges for 3 days at 37° C., fixed at the end of 3 days and the infection rate is determined by flow cytometer. The percentage of infection is determined according to the following formula:

$$Percentage of infection = \frac{Astrocyte infected with the parasite}{Total astrocyte} \times 100$$

6. Determining the Oxidative Stress by Measurement of Glutathione (GSH) Level in Astrocytes Infected with Leishmania

To determine the GSH level of infected astrocytes, cell lysate (50 ul) is suspended in DTNB (Ellman's reagent—5,5′-Dithiobis(2-nitrobenzoic acid)) (10 uL). Then, EDTA buffer solution (pH 8.2) is added to this mixture in a 96-well plate. After incubation in the dark at 37° C. for 30 minutes, absorbance is measured colorimetrically at a wavelength of 412 nm using a UV spectrophotometer (Thermo Scientific, Finland). The results are expressed as μmol/g protein.

### REFERENCES

[1]. Svobodova, M., Alten, B., Zidkova, L., Dvorak, V., Hlavackova, J., Myskova, J., . . . Volf, P. (2009). Cutaneous leishmaniasis caused by Leishmania infantum transmitted by Phlebotomus tobbi. Int J Parasitol, 39(2), 251-256. doi.org/S0020-7519(08)00270-1 [pii]r10.1016/j.ijpara.2008.06.016
[2]. Ok, 0. Z., Balcloglu, i. C., Taylan Ozkan, A., Ozensoy, S. & Ozbel, Y. (2002). Leishmaniasis in turkey. Acta Tropica, 84, 43-48
[3]. Tanir, G., Taylan Ozkan, A., & Daglar, E. (2006). Pediatric visceral Leishmaniasis in Turkey. Pediatrics International, 48(1), 66-69. doi.org/10.1 1 1 l/j 0.1442-200X.2006.02153.x
[4]. Das, B. C., Thapa, P., Karki, R., Schinke, C., Das, S., Kambhampati, S., . . . & Evans, T. (2013). Boron chemicals in diagnosis and therapeutics. Future medicinal chemistry, 5(6), 653-676.
[5]. Cebeci, E., Yiiksel, B., & Sahin, F. (2022). Anti-cancer effect of boron derivatives on small-cell lung cancer. Journal of Trace Elements in Medicine and Biology, 70, 126923.
[6]. Argin, S., Giilerim, M., & Sahin, F. (2019). Development of antimicrobial gelatin films with boron derivatives. Turkish Journal of Biology, 43(1), 47-57.
[7]. Yilmaz, M. T. (2012). Minimum inhibitory and minimum bactericidal concentrations of boron compounds against several bacterial strains. TurkishJournalofMedicalSciences, 42(8), 1423-1429.
[8]. Sundar, S. & Chakravarty, J. 2013. Leishmaniasis: An update of current pharmacotherapy. Expert Opinion on Pharmacotherapy, 14, 53-63.
[9]. Murray, H. W., Berman, J. D., Davies, C. R. & Saravia, N. G. 2005. Advances in leishmaniasis. Lancet, 366, 1561-77.
[10]. Sundar, S., Chatterjee, M. (2006). Visceral leishmaniasis—current therapeutic modalities. Indian J Med Res, 123, 345-52.
[11]. Piscopo, T. V, & Mallia Azzopardi, C. (2007). Leishmaniasis. Postgraduate Medical Journal, 83(976), 649-57. doi.org/10.1136/pgmj 2006.047340corrl
[12]. Sundar, S., Makharia, A., More, D. K., Agrawal, G., Voss, A., Fischer, C., Bachmann, P. & Murray, H. W. 2000. Short-course of oral miltefosine for treatment of visceral leishmaniasis. Clinical Infectious Diseases, 31, 1110-1113.
[13]. Jha, T. K., Sundar, S., Thakur, C. P., Bachmann, P., Karbwang, J., Fischer, C., Voss, A. & Berman, J. 1999. Miltefosine, an oral agent, for the treatment of indian visceral leishmaniasis. New England Journal of Medicine, 341, 1795-1800.
[14]. Murray, H. W., Berman, J. D., Davies, C. R. & SARAVIA, N. G. 2005. Advances in leishmaniasis. Lancet, 366, 1561-77.
[15]. Croft, S. L. & Olliaro, P. 2011. Leishmaniasis chemotherapy-challenges and opportunities. Clinical Microbiology and Infection, 17, 1478-1483.
[16]. Ahmed, A. H. A. M., Brocchini, S. & Croft, S. L. 2012. Recent advances in development of amphotericin b formulations for the treatment of visceral leishmaniasis. Current Opinion in Infectious Diseases, 25, 695-702.
[17]. Melo, G. D., Goyard, S., Piette, L., Boissonnas, A., Combadiere, C., Machado, G. F., . . . & Lang, T. (2017). Unveiling Cerebral Leishmaniasis: parasites and brain inflammation in Leishmania donovani infected mice. Scientific reports, 7(1), 1-13.
[18]. Petersen, C. A., & Greenlee, M. H. W. (2011). Neurologic manifestations of Leishmania spp. infection. Journal of neuroparasitology, 2.
[19]. Yurdakok, K., and ince, T. (2008). A 1 adjuvanlan. (:ocuk Saghgl ve Hastallklan Dergisi. 51: 225-239
[20]. Wang Y Q, Bazin-Lee H, Evans J T, Casella C R, Mitchell T C. MPL Adjuvant Contains Competitive Antagonists of Human TLR4. Front Immunol. 2020 Oct. 16; 11:577823. doi: 10.3389/fimmu.2020.577823

What is claimed is:

1. A boron derivative used in a treatment of a cerebral Leishmaniasis disease of a neuronal origin, wherein the boron derivative has a lethal effect on astrocytes infected with Leishmania parasites, without showing a toxicity in healthy cells.

2. The boron derivative according to claim 1, wherein the boron derivative is sodium perborate tetrahydrate (SPT).

3. The boron derivative according to claim 1, wherein the Leishmania parasites are at least one selected from the group consisting of L. Arabica, L. archibaldi, L. aristedesi, L. braziliensis, L. chagasi, L. colombiensis, L. Deanei, L. donovani, L. enrietii, L. equatorensis, L. forattinii, L. garnhami, L. gerbil, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. tarentolae, L. tropica, L. turanica, and L. venezuelensis.

4. A pharmaceutical composition, comprising the boron derivative according to claim 1.

5. The pharmaceutical composition according to claim 4, further comprising:
extracellular vesicles, wherein the extracellular vesicles are obtained from the astrocytes infected with the Leishmania parasites, and
nano-carrier systems, wherein the nano-carrier systems are selected from the group consisting of the following:
emulsion systems,
biological nanoparticles and chemical nanoparticles, comprising polymeric nanoparticles and solid lipid nanoparticles,
inorganic nanoparticles, comprising metallic nanoparticles,
lipid vesicular systems, comprising liposomes, niosomes, and ethosomes,
dendrimers,
polymer-drug conjugates micelles, and
carbon nanotubes.

6. The pharmaceutical composition according to claim 4, further comprising at least one active compound selected from the group consisting of an active compound showing an antiparasitic effect and/or an active compound showing an antineoplastic effect, a binary combination thereof, and a ternary combination thereof, as an active substance.

7. The pharmaceutical composition according to claim 6, wherein the active compound showing the antiparasitic effect comprises at least one of nitazoxanide, melarsoprol, eflornithine, metronidazol, tinidazole, miltefosine, mebendazole, pyrantel pamoate, thiabendazole, diethylcarbamazine, ivermectin, niclosamide, praziquantel, albendazole, rifampin, amphotericin B, fumagillin, furazolidone, nifursemizone, nitazoxanide, ornidazole, paromomycin sulfate, pentamidine, pirimethamine, tinidazole, albendazole, mebendazole, thiabendazole, fenbendazole, triclabendazole, flubendazole, abamectin, diethylcarbamazine, ivermectin, suramin, pyrantel pamoate, levamisole, niclosamide, nitazoxanide, oxyclozanide, monepantel, derquantel, amphotericin B, urea stibamine, sodium stibogluconate, meglumine antimoniate, paromomycm, miltefosine, fluconazole, pentamidine, bisnaphthalimidopropyl (BNIP) derivatives, a binary combination thereof or a ternary combination thereof, and encapsulations thereof.

8. The pharmaceutical composition according to claim 6, wherein the active compound showing the antineoplastic effect is in combination with extracellular vesicles and/or nano-carrier systems, and comprises at least one of cyclophosphamide, ifosfamide, temozolomide, capecitabine, 5-fluorouracil, methotrexate, gemcitabine, pemetrexed, mitomycin, bleomycin, epirubicin, doxorubicin, etoposide, paclitaxel, irinotecan, docetaxel, vincristine, carboplatin, cisplatin, oxaliplatin, bevacizumab, cetuximab, gefitinib, imatinib, trastuzumab, denosumab, rituximab, sunitinib, zoledronate, abiraterone, anastrozole, bicalutamide, exemestane, goserelin, medroxyprogesterone, octreotide, tamoxifen, bendamustine, carmustine, chlorambucil, lomustine, melphalan, procarbazine, streptozocin, fludarabine, raltitrexed, actinomycin D, dactinomycin, doxorubicin, mitoxantrone, eribulin, topotecan, vinblastine, vinorelbine, afatinib, aflibercept, crizotinib, dabrafenib, interferon, ipilimumab, lapatinib, nivolumab, panitumumab, pembrolizumab, pertuzumab, sorafenib, trastuzumab emtansine, temsorilimus, vemurafenib, ibandronic acid, pamidronate, bexarotene, buserelin, cyproterone, degarelix, folinic acid, fulvestrant, lanreotide, lenalidomide, letrozole, leuprorelin, megestrol, mesna, thalidomide, vincristine, a binary combination thereof or a ternary combination thereof, and encapsulations thereof.

9. The pharmaceutical composition according to claim 4 wherein the pharmaceutical composition is configured for at least one administration method selected from the group consisting of a_parenteral administration method, an intravenous administration method, an intradermal administration method, a subcutaneous administration method, an intraperitoneal administration method, a topical administration method, an intrathecal administration method, an intranasal administration method, an intracerebroventricular administration method, an ocular administration method, a vaginal administration method, a urethral administration method, a transdermal administration method, a sublingual administration method, a subarachnoid administration method, a rectal administration method, a periodontal administration method, a perineural administration method, a peridural administration method, a periarticular administration method, an oral administration method, an intratympanic administration method, an intratumor administration method, an intrapulmonary administration method, an intrasynovial administration method, an intramuscular administration method, an intraovarian administration method, an intrameningeal administration method, an intracorporus cavemosum administration method, an intracoronary administration method, an intracerebral administration method, an epidural administration method, a cutaneous administration method, a buccal administration method, and a dental administration method.

10. The pharmaceutical composition according to claim 4, further comprising an adjuvant, wherein the adjuvant is at least one selected from the group consisting of monophosphoryl lipid A (MPL), cholesterol, CG oligonucleotide-containing aluminum hydroxide, aluminum phosphate, tocopherol, emulsion systems, or a binary thereof or more combinations thereof, and the pharmaceutical composition is used in the treatment of the cerebral Leishmaniasis disease.

11. The pharmaceutical composition according to claim 4, wherein the boron derivative is SPT.

12. The pharmaceutical composition according to claim 4, wherein in the boron derivative, the Leishmania parasites are at least one selected from the group consisting of L. Arabica, L. archibaldi, L. aristedesi, L. braziliensis, L. chagasi, L. colombiensis, L. Deanei, L. donovani, L. enrietii, L. equatorensis, L. forattinii, L. garnhami, L. gerbil, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. tarentolae, L. tropica, L. turanica, and L. venezuelensis.

13. The pharmaceutical composition according to claim 5, further comprising at least one active compound selected from the group consisting of an active compound showing an antiparasitic effect and/or an active compound showing an antineoplastic effect, a binary combination thereof, and a ternary combination thereof, as an active substance.

14. The pharmaceutical composition according to claim 5, wherein the pharmaceutical composition is configured for at least one administration method selected from the group consisting of a parenteral administration method, an intravenous administration method, an intradermal administration method, a subcutaneous administration method, an intraperitoneal administration method, a topical administration method, an intrathecal administration method, an intranasal administration method, an intracerebroventricular administration method, an ocular administration method, a vaginal administration method, a urethral administration method, a transdermal administration method, a sublingual administration method, a subarachnoid administration method, a rectal administration method, a periodontal administration method, a perineural administration method, a peridural administration method, a periarticular administration method, an oral administration method, an intratympanic administration method, an intratumor administration method, an intrapulmonary administration method, an intrasynovial administration method, an intramuscular administration method, an intraovarian administration method, an intrameningeal administration method, an intracorporus cavemosum administration method, an intracoronary administration method, an intracerebral administration method, an epidural administration method, a cutaneous administration method, a buccal administration method, and a dental administration method.

15. The pharmaceutical composition according to claim 6, wherein the pharmaceutical composition is configured for at least one administration method selected from the group consisting of a parenteral administration method, an intravenous administration method, an intradermal administration method, a subcutaneous administration method, an intraperitoneal administration method, a topical administration method, an intrathecal administration method, an intranasal administration method, an intracerebroventricular administration method, an ocular administration method, a vaginal administration method, a urethral administration method, a transdermal administration method, a sublingual administration method, a subarachnoid administration method, a rectal administration method, a periodontal administration method, a perineural administration method, a peridural administration method, a periarticular administration method, an oral administration method, an intratympanic administration method, an intratumor administration method, an intrapulmonary administration method, an intrasynovial administration method, an intramuscular administration method, an intraovarian administration method, an intrameningeal administration method, an intracorporus cavemosum administration method, an intracoronary administration method, an intracerebral administration method, an epidural administration method, a cutaneous administration method, a buccal administration method, and a dental administration method.

16. The pharmaceutical composition according to claim 7, wherein the pharmaceutical composition is configured for at least one administration method selected from the group consisting of a parenteral administration method, an intravenous administration method, an intradermal administration method, a subcutaneous administration method, an intraperitoneal administration method, a topical administration method, an intrathecal administration method, an intranasal administration method, an intracerebroventricular administration method, an ocular administration method, a vaginal administration method, a urethral administration method, a transdermal administration method, a sublingual administration method, a subarachnoid administration method, a rectal administration method, a periodontal administration method, a perineural administration method, a peridural administration method, a periarticular administration method, an oral administration method, an intratympanic administration method, an intratumor administration method, an intrapulmonary administration method, an intrasynovial administration method, an intramuscular administration method, an intraovarian administration method, an intrameningeal administration method, an intracorporus cavemosum administration method, an intracoronary administration method, an intracerebral administration method, an epidural administration method, a cutaneous administration method, a buccal administration method, and a dental administration method.

17. The pharmaceutical composition according to claim 8, wherein the pharmaceutical composition is configured for at least one administration method selected from the group consisting of a parenteral administration method, an intravenous administration method, an intradermal administration method, a subcutaneous administration method, an intraperitoneal administration method, a topical administration method, an intrathecal administration method, an intranasal administration method, an intracerebroventricular administration method, an ocular administration method, a vaginal administration method, a urethral administration method, a transdermal administration method, a sublingual administration method, a subarachnoid administration method, a rectal administration method, a periodontal administration method, a perineural administration method, a peridural administration method, a periarticular administration method, an oral administration method, an intratympanic administration method, an intratumor administration method, an intrapulmonary administration method, an intrasynovial administration method, an intramuscular administration method, an intraovarian administration method, an intrameningeal administration method, an intracorporus cavemosum administration method, an intracoronary administration method, an intracerebral administration method, an epidural administration method, a cutaneous administration method, a buccal administration method, and a dental administration method.

18. The pharmaceutical composition according to claim 5, further comprising an adjuvant, wherein the adjuvant is at least one selected from the group consisting of MPL, cholesterol, CG oligonucleotide-containing aluminum hydroxide, aluminum phosphate, tocopherol, emulsion systems, or a binary thereof or more combinations thereof, and the pharmaceutical composition is used in the treatment of the cerebral Leishmaniasis disease.

19. The pharmaceutical composition according to claim 6, further comprising an adjuvant, wherein the adjuvant is at least one selected from the group consisting of MPL, cholesterol, CG oligonucleotide-containing aluminum hydroxide, aluminum phosphate, tocopherol, emulsion systems, or a binary thereof or more combinations thereof, and the pharmaceutical composition is used in the treatment of the cerebral Leishmaniasis disease.

20. The pharmaceutical composition according to claim 7, further comprising an adjuvant, wherein the adjuvant is at least one selected from the group consisting of MPL, cholesterol, CG oligonucleotide-containing aluminum hydroxide, aluminum phosphate, tocopherol, emulsion systems, or a binary thereof or more combinations thereof, and the pharmaceutical composition is used in the treatment of the cerebral Leishmaniasis disease.
